(19) United States
(12) Patent Application Publication
(10) Pub. No.: US 2022/0143656 A1
(43) Pub. Date: May 12, 2022

(54) Method and Device for Cleaning and Disinfecting a Textile Surface and a Disinfection Box

(71) Applicant: MattPro GmbH, Benz OT Labomitz (DE)

(72) Inventor: Eberhard Grzeskowiak, Usedom (DE)

(21) Appl. No.: 17/517,682

(22) Filed: Nov. 3, 2021

(30) Foreign Application Priority Data
Nov. 9, 2020 (DE) 102020129466.2

(51) Int. Cl. B08B 5/04; B08B 7/00; A47L 7/00; A47L 9/28; A61L 2/10

(52) U.S. Cl. CPC B08B 5/04; B08B 7/0057; A47L 7/0061; A47L 9/28; A61L 2202/17; A61L 2/10; A61L 2202/11; A61L 2202/14; A47L 7/0004

(57) ABSTRACT

A method and a device for cleaning and disinfecting a textile surface by means of UVC radiation. Furthermore, the invention relates to a disinfection box (2) generating UVC radiation. The textile surface to be cleaned is imaged before and after cleaning. On the basis of the surface area of the textile surface a duration of treatment is set. The disinfection box (2) comprises at least one UVC light source (4) of elongated shape as well as a ballast (5) to control the same. The cleaning device for cleaning and disinfecting a textile surface comprises, in addition to the disinfection box (2), a commercially available industrial vacuum cleaner (1), on the front side of which the disinfection box (2) is arranged.

### CROSS REFERENCE TO RELATED APPLICATIONS

This application claims the priority of DE 102020129466.2 filed on 2020 Nov. 9; this application is incorporated by reference herein in its entirety.

### BACKGROUND

The invention is related to a method for cleaning and disinfecting by means of which a textile surface contaminated with biological allergens can be cleaned using UVC light as well as a vacuum cleaner. Furthermore, the invention relates to a disinfection box as well as a device for cleaning comprising said disinfection box. The invention is applicable in particular for cleaning mattresses, upholstered furniture or carpets.

Mattresses, as well as upholstered furniture or carpets, are usually contaminated with dead skin cells and dust mites. During their life, mites produce about 200 times their weight in excrements, turning a mattress into a terrain with a heavy concentration of allergens.

From the state of the art it is known to kill bacteria, germs or microorganisms using UV (Ultra Violet) radiation. Bacteria are particularly deactivated by irradiating them with UVC radiation at a wavelength of 254 nm.

A method and a device for cleaning mattresses, upholstered furniture and other textile objects is shown in DE 41 39 199 A1. The document shows a type of vacuum cleaner brush with an integrated vibration generating device which subjects the surface to be cleaned to mechanical vibrations so that adhering dirt particles are loosened or smashed. With the aid of a vacuum generation device, the contaminants are extracted. In addition, disinfection with ultraviolet radiation in the wavelength range of 273 nm can be provided.

DE 195 19 494 C1 describes a method for cleaning and removing house dust mites from mattresses, during which a toxic gas, namely carbon dioxide, is introduced into the mattress in addition to a vacuum cleaning treatment. However, a container with the gas must always be carried along for this purpose, which is unreasonably costly for routine cleaning of beds in a hotel, for example.

In the case of cleaning beds in a hotel room it furthermore may happen that the cleaning staff is cleaning and disinfecting, respectively, the mattresses less thoroughly due to a lack of time.

Another disadvantage of the above-mentioned cleaning methods is a lack of consideration for different disinfection requirements, so that, for example, large mattresses are cleaned in the same period of time as small mattresses.

### SUMMARY

The invention is based on the task of avoiding the above-mentioned disadvantages, whereby a method and a device for cleaning and disinfecting textile surfaces is to be provided, which enables both dry and environmentally friendly antibacterial cleaning without the use of chemical cleaning agents, whereby it is to be ensured that an effective disinfection adapted to the size of the respective textile surface is carried out.

The task is solved by a method for cleaning and disinfecting a textile surface by means of UVC radiation having the features according to claim 1, a disinfection box for disinfecting a textile surface having the features according to claim 7, and a device for cleaning and disinfecting a textile surface by means of UVC radiation according to claim 10. Useful embodiments of the invention can be found in the remaining claims.

### DETAILED DESCRIPTION

According to the disclosure, a method is provided for cleaning a textile surface and disinfecting it by means of UVC radiation using a hand-held vacuum cleaner with a disinfection box arranged thereon, the method comprising capturing images of the textile surface, automated determining the required time for cleaning as well as recording of the actual cleaning time.

In a first step of the method an image of the whole textile surface to be cleaned is being captured by means of a camera, which camera may be part of a control unit. Furthermore, an area size of the textile surface to be cleaned is determined. This determination of the area size may be performed automated by the control unit, e.g. on the basis of predefined characteristics, or it may be performed by manually entering the size value into the control unit by a housekeeper carrying out the cleaning.

On the basis of the identified area size a minimum required duration of treatment of the textile surface with UVC radiation for complete disinfection is determined by the control unit.

Hereafter, the textile surface is cleaned using the hand-held vacuum cleaner and at the same time the textile surface is disinfected using UVC radiation, said cleaning and disinfecting being performed for at least the minimum necessary treatment time previously determined. During treatment, an image of a partial area of the textile surface is captured at predetermined time intervals by means of a second camera arranged in or on the disinfection box. The images are stored in a data storage device of the disinfection box or transmitted to a data storage device of the control unit. These time intervals can be in the millisecond range, i.e. a film is created, or may be up to several minutes, so that only individual images are captured and stored. It is also conceivable to capture only two images per cleaning process with the second camera.

After the minimum necessary treatment time has elapsed, the person cleaning is signaled optically and/or acoustically, e.g. by the control unit, that cleaning can be terminated.

Finally, an image of the whole, now cleaned textile surface is captured by the first camera.

These two images of the whole textile surface captured by the first camera are stored together with the previously determined minimum necessary treatment time and the actual cleaning time as well as a designation, e.g. name or identification number, of the person performing the cleaning, e.g. in a data storage device of the control unit.

The control unit may be a commercially available smartphone with camera, wherein a dedicated cleaning control software (a so-called app) is installed on the smartphone.

Since the minimum necessary treatment time is determined by the control unit on the basis of the size of the textile surface to be cleaned, sufficient and effective disinfection is always guaranteed. The permanent image capture by means of the second camera ensures later that the cleaner actually worked on the textile surface during the entire predetermined necessary treatment time.

Thus, in combination with the data on cleaning time as well as the images of the textile surface before and after cleaning, quality assurance is possible.

By dispensing with additional chemical cleaning agents, there is no waste and no environmental pollution. Also, due to the UV-based disinfection, chemically resistant pathogens are killed.

The method according to the invention can further be designed in such a way that, in addition to the above-mentioned data, the location of the cleaned textile surface is additionally recorded and stored using GPS data, as well as the current date and time. For this purpose the data storage device of the disinfection box, preferably however the data storage device of the control unit, is used.

Furthermore it may be provided that all stored data, i. e. the data stored in the data storage device of the disinfection box as well as in the data storage device of the control unit, are transferred wirelessly to a server at least every 24 hours, e.g. at the end of a working day, or after each cleaning performed. Said transfer may be conducted via an internet connection, e. g. by means of WLAN. For this purpose, the control unit comprises a communication module for connecting to the internet.

The server preferably is connected to the internet, wherein an e. g. graphical user interface (GUI) enables access via the Internet to the stored data of the server. Thus, in an advantageous way, the cleaning can be checked from any place where a connection to the Internet is available.

According to an embodiment, a necessary replacement of the UVC lamp generating the UVC radiation due to reaching the maximum service life is signaled optically and/or acoustically. Said signaling may be achieved by means of a signaling device arranged on or in the disinfection box or by a signal generated by the control unit. This ensures that the UVC radiation used for disinfection always has the required intensity.

If appropriate, it may be advisable to treat the textile surface with a liquid or gaseous disinfectant before or after cleaning.

In addition or alternatively, the textile surface can be cooled to a temperature below −78.5° C., for example with dry ice, and thus sterilized. The dry ice can be applied to the textile surface in the form of carbon dioxide snow, e.g. by means of a spray lance, so that the surface is “shock-frozen” at around −79° C.

The disinfection box according to the disclosure for disinfecting a textile surface comprises at least one, preferably elongated, light source for emitting UVC light in the direction of the textile surface, a ballast for controlling the power and light frequency of the light source, a camera directed at the textile surface, a data storage device connected to the camera, and a wireless interface connected to the data storage device.

One advantage is that, thanks to a regulating ballast, the UVC radiation used for disinfection is always irradiated onto the textile surface with the required intensity and wavelength. Intensity fluctuations during a cleaning process or with increasing age of the UVC light source are thus excluded, so that effective disinfection in accordance with the task is ensured.

The wireless interface can be a Bluetooth module or a WLAN module. By means of the wireless interface, the data or images captured by the disinfection box can be transferred to a control unit and/or via the Internet to a dedicated server.

In addition it may be provided to control the disinfection box via the wireless interface, in particular to switch on and off the UVC light source.

Preferably, a gas discharge tube is used as the UVC light source. Here, the disinfection box additionally exhibits a shatter protection, which protects against flying glass splinters in the event of a breakage of the gas discharge tube. The UVC light source itself is equipped with a special housing, which has metal reflectors on its inside. Thus, the reflection of UV light at the reflectors allows for a maximum as well as directed UV emission.

The UVC light source exhibits preferably a power of at least 27 W, if possible of minimum 42 W, in particular of at least 59 W.

According to one embodiment, the disinfection box comprises two elongated UVC light sources, in particular two gas discharge tubes.

Further it may be provided that the UVC light source during operation emits UVC radiation in a wave length range of less than 275 nm, preferably from essentially 253 nm. Due to this short-wave, high-energy UV radiation, a very efficient germicidal effect is achieved.

Furthermore, the disinfection box may comprise a display unit, by means of which for example a required replacement of the UVC light source and/or a duration of cleaning (i. e. how much time actually elapsed for the treatment of the mattress) may be indicated.

The device for cleaning according to this disclosure comprises a commercially available hand-held vacuum cleaner, at the front of which a disinfection box with the features described above is arranged. Preferably, an industrial type vacuum cleaner is used, which exhibits an enhanced suction power in comparison to a household vacuum cleaner. The device for cleaning may be used in an advantageous manner for a mobile cleaning of mattresses in e. g. hotel rooms due to its small dimensions and low weight. By dispensing with additional gas tanks containing germicidal gas, the cleaning device is very handy and can therefore be used and carried from room to room even by a petite cleaner.

Furthermore, the device for cleaning may comprise nozzles fluidic connected to a tank containing a liquid. These nozzles are directed to the textile surface when using the device for cleaning as intended. This allows the textile surface to be cleaned to be sprayed with a liquid suitable for killing bacteria, germs or other microorganisms.

### BRIEF DESCRIPTION OF THE DRAWINGS

In the following the invention will be explained in detail on the basis of an exemplary embodiment, wherein identical or similar features are provided with the same reference numerals. For this, in schematic representation show the

FIG. 1 an embodiment of the device for cleaning and disinfecting of a textile surface in oblique view from above; and

FIG. 2 an embodiment of the disinfection box in a longitudinal view.

### DETAILED DESCRIPTION OF THE PREFERRED EMBODIMENTS

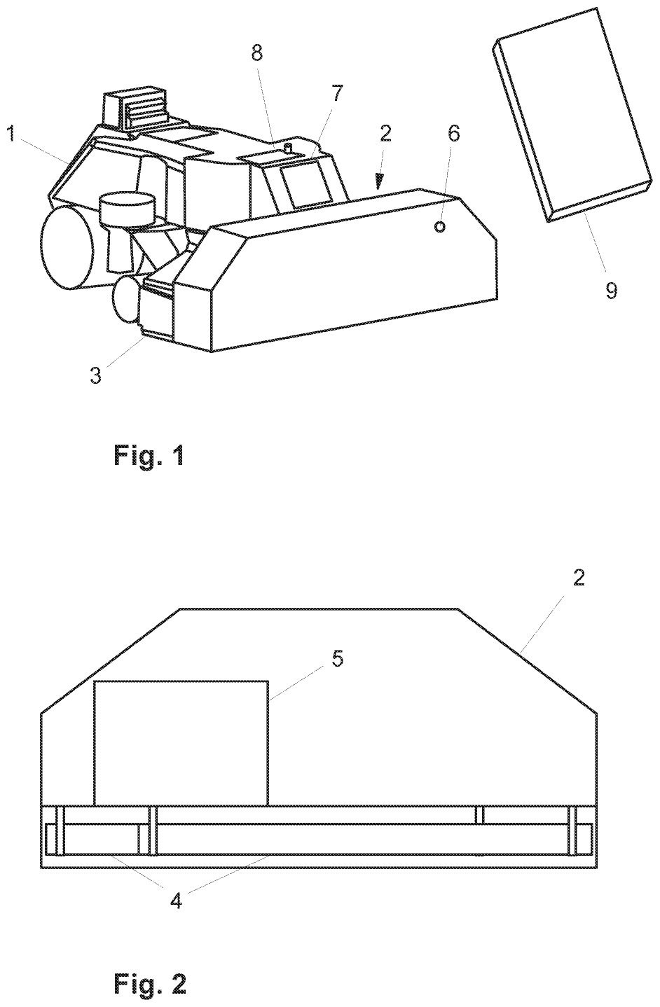

The device for cleaning and disinfecting a textile surface according to FIG. 1 comprises the hand-held vacuum cleaner 1, on the end face of which the disinfection box 2 is arranged in such a way that it—or its elongated UVC light source (not shown)—is aligned parallel to the suction nozzle 3 of the hand-held vacuum cleaner 1, the width of the area on the textile surface irradiated by the UVC light source corresponding essentially to the width of the area covered by the suction nozzle 3.

The disinfection box 2 comprises the camera, which is located at the front side of the disinfection box 2 and is directed obliquely downward. The disinfection box 2 further comprises the display unit 7 as well as the wireless interface 8 located at the upper side of the disinfection box 2, the wireless interface 8 being a bluetooth module in this example.

The smartphone 9 is used as control unit, on which is installed a software (app) for determining and displaying the required duration of treatment and for communicating with the wireless interface 8 and a server connected to the Internet (not shown).

The disinfection box 2 for disinfecting a textile surface shown in FIG. 2 comprises a first rod-shaped UVC light source 4 and a second rod-shaped UVC light source 4 (behind the first UVC light source in the figure), the UVC light sources 4 in this example being gas discharge tubes which are controlled by the ballast 5 so that they emit UV light of wavelength 253 nm with a power of 42 W each.

### LIST OF REFERENCE NUMERALS

1 hand-held vacuum cleaner
2 disinfection box
3 suction nozzle
4 UVC light source
5 ballast
6 camera
7 display unit
8 wireless interface
9 smartphone

1. A method for cleaning and disinfecting a textile surface using a hand-held vacuum cleaner (1) at which is attached a disinfection box (2) generating UVC radiation, the method comprising the following steps:
capturing an image of the whole surface area of the textile surface by use of a first camera;
determining a surface area size of the textile surface to be cleaned;
determining a minimum required duration of irradiating the textile surface with UVC radiation in order to achieve comprehensive disinfection of the textile surface;
cleaning of the textile surface by use of the hand-held vacuum cleaner (1) and disinfecting the textile surface for at least the minimum required duration of irradiating by use of UVC radiation,
wherein at predetermined time intervals an image of a partial region of the textile surface is captured by means of a second camera (6) arranged in or on the disinfection box (2) and stored in a data storage device of the disinfection box (2);
capturing of an image of the whole surface area of the textile surface as cleaned by use of the first camera;
saving a duration of the cleaning and a designation of the person performing the cleaning.

2. The method according to claim 1, characterized in that prior to or after the cleaning a position determined using GPS as well as the current date and time are saved.

3. The method according to claim 1, characterized in that a smartphone (9) comprising the first camera is used for capturing the image of the whole surface area of the textile surface, wherein the minimum required duration of irradiating the textile surface is determined by use of a software application running on the smartphone (9).

4. The method according to claim 1, characterized in that data stored in the data storage device of the disinfection box (2) is transferred wirelessly to a server at least every 24 hours.

5. The method according to claim 1, characterized in that a necessary exchange of the UVC light source (4) due to reaching the maximum service life of said UVC light source (4) generating the UVC radiation is signaled optically and/or acoustically.

6. The method according to claim 1, characterized in that the textile surface is treated using a liquid or gaseous germicide prior to or after the cleaning.

7. A disinfection box for disinfecting a textile surface, comprising
at least one UVC light source (4) of elongated shape for emitting UVC light in the direction of the textile surface;
a ballast (5) for controlling the light power and light frequency of the UVC light emitted by the UVC light source (4);
a camera (6) directed towards the textile surface;
a data storage device connected to the camera (6); and
a wireless interface (8) which is connected to the data storage device.

8. The disinfection box according to claim 7, characterized in that the UVC light source (4) of elongated shape for emitting UVC light is a gas discharge tube, wherein the disinfection box additionally comprises a splinter shield arranged at least partially around the gas discharge tube.

9. The disinfection box according to claim 7, characterized in that the UVC light source (4) is designed to emit UVC light having a wavelength of substantially 253 nm at a power of at least 27 watts.

10. A device for cleaning and disinfecting a textile surface comprising a hand-held vacuum cleaner (1), at the front face of which is arranged a disinfection box according to claim 7.
